(19) United States
(12) Patent Application Publication
(10) Pub. No.: US 2019/0099280 A1
(43) Pub. Date: Apr. 4, 2019

(54) SYSTEMS AND METHODS FOR THE RETRIEVAL GALLBLADDER IMPLANTS

(71) Applicant: LithiBlock Ltd., Peduel (IL)

(72) Inventor: Shmuel BEN MUVHAR, Peduel (IL)

(73) Assignee: LithiBlock Ltd., Peduel (IL)

(21) Appl. No.: 16/086,323

(22) PCT Filed: Mar. 28, 2017
(86) PCT No.: PCT/IL2017/050384

§ 371 Date: Sep. 19, 2018

Related U.S. Application Data
(60) Provisional application No. 62/313,830, filed on Mar. 28, 2016.

(51) Int. Cl. A61F 2/95; A61F 2/04

(52) U.S. Cl. CPC A61F 2/95; A61F 2002/9528; A61F 2002/041; A61F 2/04

(57) ABSTRACT

An implant retrieval system for retrieving a previously deployed implant in a patient's body lumen, comprising: an outer catheter with a lumen disposed therethrough along a central axis of the outer catheter and configured with a diameter smaller than a diameter of the deployed implant; and, a capture device configured to slide within the lumen of the outer catheter and including at least one capturing arm, outwardly expandable from the central axis, located at a distal end of the capture device, wherein the at least one capturing arm engages the previously deployed implant for withdrawing the implant into the outer catheter, while the diameter of the outer catheter, which is smaller than the deployed implant, causes the implant to collapse while being withdrawn by the capture device.

### RELATED APPLICATIONS

This application claims the benefit of priority under Article 8 PCT and 35 USC 119(e) of U.S. Provisional Patent Application No. 62/313,830 filed Mar. 28, 2016, this application is also related to pending U.S. patent application Ser. No. 15/147,080 filed May 5, 2016 and PCT Patent Application No. PCT/IL2015/051051 filed Oct. 27, 2015, the contents of all of which are incorporated by reference herein in their entireties.

### FIELD AND BACKGROUND OF THE INVENTION

The present invention relates generally to treating medical conditions involving ducts and/or body lumens, and particularly to retrieving previously implanted gallbladder implants.

“Stones” in the gallbladder and bile ducts are found in the entire population, some of them being asymptomatic, and some—symptomatic. In the U.S., 10-15% of the adult population (more than 20 million people) suffer from bile duct stones (about 20% of the population above 65 years of age suffer from gallstones), with more than two million new cases diagnosed annually, and more than 1,800,000 cholecystectomy procedures performed annually. Patients with gallstones are classified according to three groups: symptomatic, asymptomatic and those suffering from complications caused by the gallstones, such as cholecystitis, pancreatitis or obstructive jaundice.

### SUMMARY OF THE INVENTION

There is provided in accordance with an aspect, an implant retrieval system for retrieving a previously deployed implant in a patient's body lumen, comprising: an outer catheter with a lumen disposed therethrough along a central axis of the outer catheter; and, a capture device configured to slide within the lumen of the outer catheter and including at least one capturing arm, outwardly expandable from the central axis, located at a distal end of the capture device.

In some embodiments of the invention, the system further comprises an inner catheter configured to slide within a lumen of the catheter device.

In some embodiments of the invention, the system further comprises a guide wire configured to slide within a lumen of the inner catheter.

In some embodiments of the invention, the at least one capturing arm has a rounded hook shape configured to latch onto the implant.

In some embodiments of the invention, the system further comprises a plurality of capturing arms each face in the same direction.

In some embodiments of the invention, the system further comprises a plurality of capturing arms, at least one capturing arm facing in an opposite direction.

In some embodiments of the invention, the at least one capturing arm is configured with at least one of a flat, rounded and curved shape.

In some embodiments of the invention, at least one capturing arm is longer than another at least one other capturing arm.

In some embodiments of the invention, the distal end of the capture device is configured with an expansion enabling slot.

In some embodiments of the invention, an enlarged end is disposed on a distal end of the inner catheter.

In some embodiments of the invention, the enlarged end is conical shaped.

In some embodiments of the invention, the enlarged end is disposed on the inner catheter to proximally of the at least one capturing arm such that advancement of the inner catheter distally causes the enlarged end to mechanically and reversibly expand the at least one capturing arm outward from the central axis.

In some embodiments of the invention, the enlarged end has a diameter at least as large as the outer catheter.

In some embodiments of the invention, the capturing device is constructed of an elastic material.

In some embodiments of the invention, the implant retrieval system is at least 5 F. In some embodiments of the invention, the implant retrieval system is between 7 F and 8.5 F.

There is provided in accordance with an aspect, a method for retrieving a previously deployed implant from a patient, comprising: navigating to the previously deployed implant, an implant retrieval system, comprising an outer catheter with a lumen disposed therethrough along a central axis of the outer catheter; and, a capture device configured to slide within the lumen of the outer catheter and including at least one capturing arm, outwardly expandable from the central axis, located at a distal end of the capture device; pulling back the outer catheter to expose the at least one capturing arm of the capture device; latching the at least one exposed capturing arm onto the previously deployed implant; and, withdrawing the previously deployed implant and the capture device from the patient.

In some embodiments of the invention, the at least one capturing arm automatically expands when exposed by pulling back the outer catheter.

In some embodiments of the invention, the method further comprises mechanically enlarging the at least one capturing arm of the capture device using an enlarged end of an inner catheter disposed in a lumen of the capture device.

In some embodiments of the invention, the method further comprises withdrawing from the patient the remainder of the implant retrieval system after withdrawing the previously deployed implant and the capture device

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example, and not necessarily to scale, and are for purposes of illustrative discussion of embodiments of the invention. In this regard, the description taken with the drawings makes apparent to those skilled in the art how embodiments of the invention may be practiced.

In the drawings:

FIG. 1 is a block diagram of a gallbladder implant retrieval system, in accordance with an exemplary embodiment of the invention;

FIGS. 2A-2B are side views of exemplary capture arm configurations of a capture device, in accordance with an exemplary embodiment of the invention;

FIGS. 3A-3B are side views of exemplary capture devices in a catheter, in accordance with an exemplary embodiment of the invention;

FIGS. 4A-4C are frontal views of a distal end of the capture device, in accordance with exemplary embodiments of the invention;

FIG. 5 is a side view of a gallbladder implant retrieval system with a capture device partially within a catheter, in accordance with an exemplary embodiment of the invention;

FIGS. 6A-6B are side views of different capture arm configurations of an implant retrieval system, in accordance with exemplary embodiments of the invention;

FIG. 7 is a close-up view of a portion of an implant hooked by a capture arm of a capture device, in accordance with an exemplary embodiment of the invention;

FIG. 8 is a close-up view of a portion of an implant hooked by a capture arm of a capture device, in accordance with an exemplary embodiment of the invention;

FIGS. 9A-9C show a method of retrieving a gallbladder implant, in accordance with an exemplary embodiment of the invention;

FIG. 10 is a flowchart of a method of retrieving a gallbladder implant, in accordance with an exemplary embodiment of the invention; and,

FIGS. 11A and 11B depict a method of retrieving a gallbladder implant through a needle, in accordance with an exemplary embodiment of the invention.

### DETAILED DESCRIPTION

The present invention relates generally to treating medical conditions involving ducts and/or body lumens, and particularly to retrieving previously implanted gallbladder implants.

Before explaining at least one embodiment of the invention in detail, it is to be understood that the invention is not necessarily limited in its application to the details of construction and the arrangement of the components and/or methods set forth in the following description and/or illustrated in the drawings. The invention is capable of other embodiments or of being practiced or carried out in various ways. It should also be understood that while the various devices and methods described herein pertain to the biliary system, particularly the gallbladder, it is conceived that the devices and methods are possibly usable with other body lumens and/or organs such as within the cardiovascular and/or biliary systems.

It should also be understood that as used herein, “proximal” or “proximally” means closest to an attending medical professional (e.g. performing a procedure where the system is being inserted into a patient) or in a direction towards the outside of the patient's body and “distal” or “distally” means closest to the implant in a body lumen or in direction moving further inside the patient's body. Description of systems and/or devices herein, when referencing proximal or distal directions, is in the context of third person's view of a system deployed in a patient body lumen. For example, the distal end of the implant retrieval system is the end closest to the gall bladder (farthest into the patient) when the implant retrieval system is inserted into the patient to retrieve a previously deployed implant.

Generally, implant retrieval systems are described herein which are navigated through a patient's body to retrieve a previously implanted and/or deployed implant device, for example, implants which are used in the cardiovascular or biliary system. The implant retrieval systems described herein are capable of retrieving already deployed implants while simultaneously collapsing the deployed (and previously expanded) implants to facilitate retrieval. In some embodiments, the implant retrieval systems are navigated using medical imaging and/or endoscopy and/or catheters. Retrieval of a previously deployed implant is desirable for any number of reasons including, an error in original implantation (e.g. at the wrong site or location, in the wrong orientation, not fully deployed), a negative body response to the implant (e.g. inflammation, infection, pain), and to remove whatever debris has been collected by the implant (e.g. gall stones).

Various embodiments of implant retrieval systems are described herein which include different capturing arm embodiments configured with arms of varying size and/or shape and/or orientation, including with respect to other arms in the same device embodiment. Also described below are device embodiments which provide different methods of operation, for example including an expansion slot and/or an enlarged end.

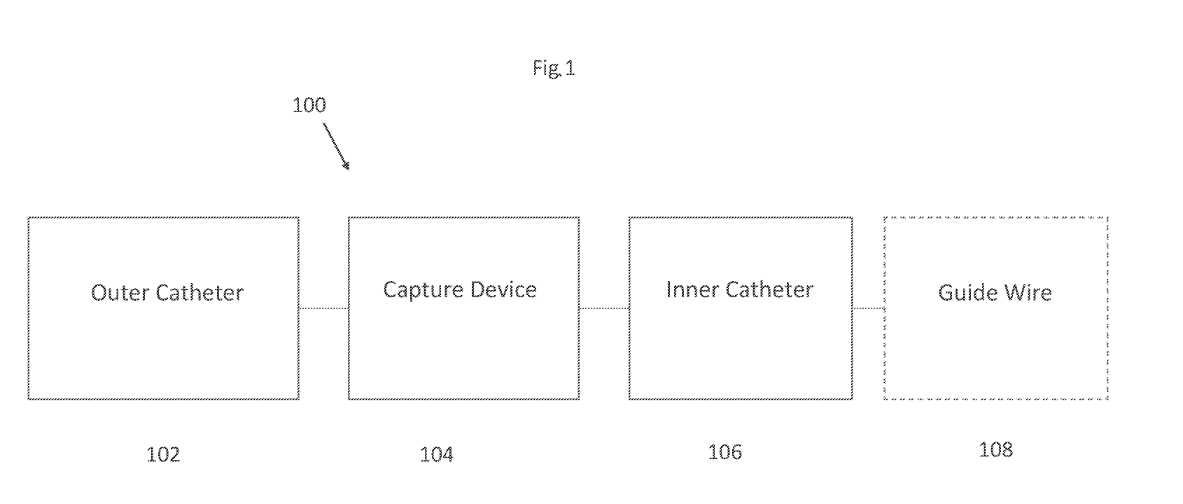

FIG. 1 is a block diagram of a gallbladder implant retrieval system 100, in accordance with an exemplary embodiment of the invention. In some embodiments of the invention, the implant retrieval system 100 comprises an outer catheter 102, a capture device 104 and optionally, an inner catheter 106 and/or a guide wire 108. The implant retrieval system 100 is configured to be inserted into a body lumen of a patient and navigated through the body to an implant, which is to be retrieved by the implant retrieval system 100. In some embodiments of the invention, the implant retrieval system 100 is navigated through the body using medical imaging. In some embodiments of the invention, the guide wire 108 is used to assist with the navigating of the implant retrieval system 100 through the patient's body to the implant to be retrieved.

In some embodiments of the invention, the capture device 104 is inserted into the patient's body within a lumen of the outer catheter 102. Additionally and/or optionally, the inner catheter 106 is positioned within a lumen of the capture device 104. Additionally and/or optionally, the guide wire 108 is positioned within the inner catheter 106 and/or the capture device 104.

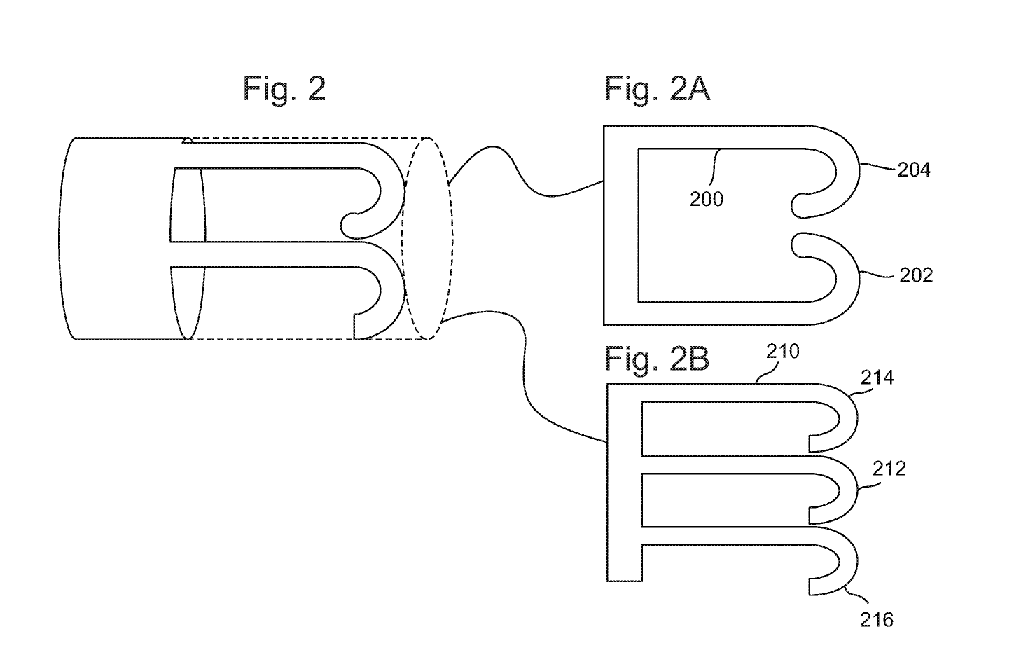

The capture device 104 is generally tubular, is configured to be slidable within the outer catheter 102 and is provided with at least one capturing arm, shown and described in more detail with respect to FIGS. 2A-9C, at a distal end of the capture device adapted for hooking and/or latching on to an implant to retrieve the implant from the patient's body.

In an embodiment of the invention, the capture device is constructed of an elastic material such as an elastic metal and/or plastic. In some embodiments, the capture device behaves super elastically or plastically. In some embodiments, the catheter sizes are 5 F and greater. In some embodiments, the catheters are between 7 F and 8.5 F. These sizes are by way of example only. It should also be noted that, in some embodiments, the outer catheter 102 has a diameter which is smaller than the general diameter of the deployed (already expanded in situ) implant which is being captured. The smaller diameter of the outer catheter 102 effectuates the collapse of the larger diameter, deployed implant being retrieved, as the implant is being withdrawn into the lumen of the outer catheter 102.

FIGS. 2A-2B are side views of exemplary capturing arm configurations of a capture device 104, in accordance with an exemplary embodiment of the invention. FIG. 2A is a side view of a capturing arm configuration 200 where there are two capturing arms 202, 204 which are configured with hooks at their distal ends for latching on to an implant and where the hooks face towards each other, that is, in opposing directions.

FIG. 2B shows a configuration 210 where there are a plurality of capturing arms 212, 214, 216 with hooks at their distal ends which all face in the same direction.

It should be understood that the configurations 200, 210 shown in FIGS. 2A-2B are by way of example only. There could be one, two or more arms. In some embodiments, there is more than one hook per arm. While individual arm dimensions can vary, in some embodiments, at least one arm is 0.5-20 mm in length (“length” extending in the major axis of the arm). In some embodiments, at least one arm is 0.05-0.8 mm in width (“width” extending in the minor axis of the arm, transverse to the length). In some embodiments, the hook portion of the arm has a radius of 0.05-10 mm.

In some embodiments, the radius of the hook varies from arm to arm, even if there are a plurality of arms in the configuration. In some embodiments, not all of the hooks face in the same direction. In some embodiments, the arms are different lengths and/or are variable in length. In some embodiments, the arms are biased at different angles, for example they may not be parallel to one another. In some embodiments, the arms are evenly spaced around the circumference of distal end of the capture device (i.e. at same arc angles with respect to each other). In some embodiments, at least some of the arms are clustered together. In some embodiments, at least some of the arms are arranged around the circumference of the distal end unevenly. As described elsewhere herein, some embodiments have a plurality of arms in the same device, but which have arms with different lengths, orientations, angles of curvature (for the hooks), sizes, etc. with respect to each other.

It should be understood that these capturing devices are used to retrieve expanded implants which are likely not expanded in any kind of regular or predictable manner and are also most likely not directly abutting a wall of a body lumen against which the implant can be pressed to facilitate implant removal. Using capturing arms of varying configurations may enhance the probability and/or ease of capture.

Another aspect of some of the capturing arm configurations described herein includes the “closed” nature of the rounded, hook-shaped capturing arms when being advanced distally, but the “open” nature of the same hook-shaped capturing arms when being retracted in a proximal direction. While moving in the distal direction, this configuration assists with the movement of the capturing arms through and into the implant from where the implant can be engaged, while also providing a measure of safety for being rounded and not sharp at the distal end (e.g. in case the hook pokes a body wall while being moved around). While moving in the proximal direction, the open nature of the hook-like configuration allows for latching/catching/engaging of the hook on the implant, for example a loop/petal structure of the implant. An additional feature of this configuration is that the latching/catching/engaging of the implant can be achieved without the need of clamping or the application of a gripping pressure.

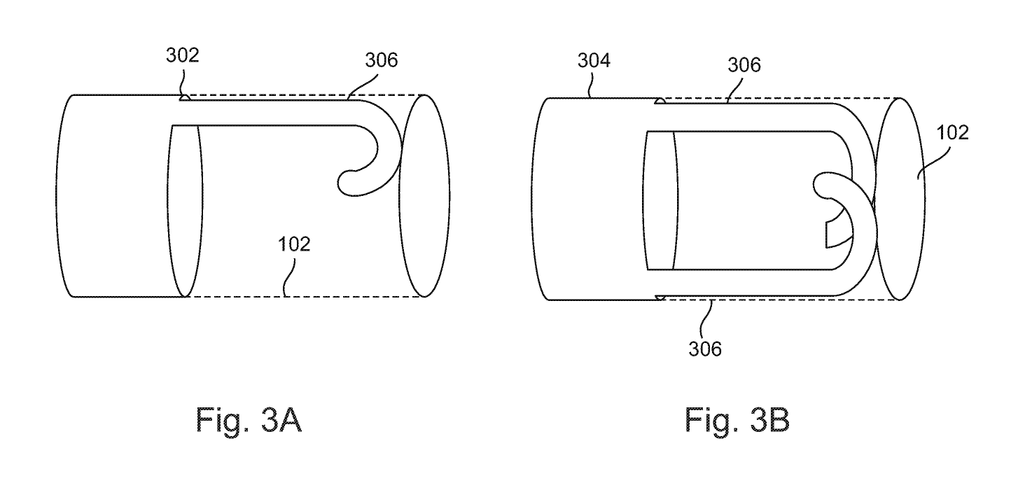

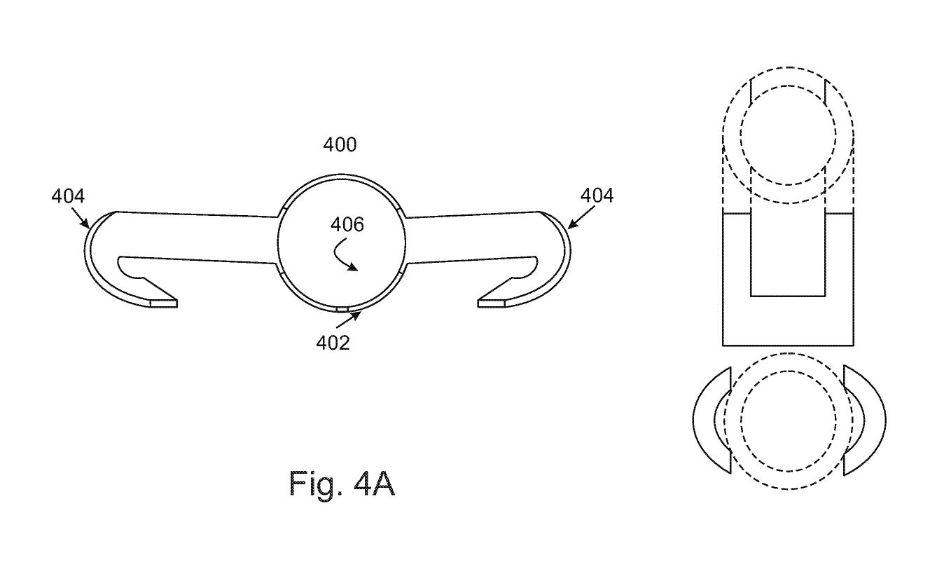

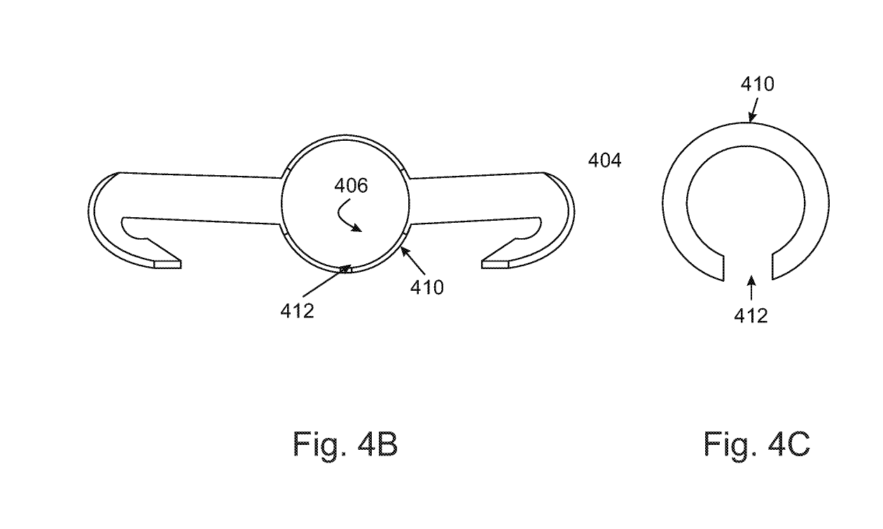

FIGS. 3A-3B are side views of exemplary capture devices 302, 304 in an outer catheter 102, in accordance with an exemplary embodiment of the invention. As shown in FIGS. 3A-3B, the capture devices 302, 304 are folded and/or compressed to fit them within the lumen of the outer catheter 102 for navigation through the patient's body and eventual deployment for retrieval of an implant. In some embodiments of the invention, the arms 306 of the capture devices 302, 304 expand when the capture devices 302, 304 are extended out of the outer catheter 102, for example as shown in FIG. 4A. In some embodiments, the arms of the capture devices described herein (not necessarily just for exemplary capture devices 302, 304) are flat, not round, in cross-section to reduce the amount of space they take up inside the lumen of the navigating catheters when they are compressed together during transit/navigation. Optionally, the arms are curved, to correspond with the curvature of the outer catheter 102 (such as shown in FIG. 4A).

FIGS. 4A-4C are frontal views of a distal end 402 of the capture device 400, in accordance with exemplary embodiments of the invention. In an embodiment of the invention, FIG. 4A shows arms 404 which have expanded radially (in relation to a central axis 406 of the device 400) upon deployment of the device 400 from the outer catheter 102. As described elsewhere herein, the arms 404 can have varying configurations (e.g. shapes and/or sizes and/or orientations) but are shown in this example as being rounded with hooks. In an embodiment of the invention, the rounded shape facilitates the insertion of the capturing arm 404 into the implant (the rounded shape avoids the arm catching on the implant and/or body tissues as the capture device 400 is advanced distally), while the hook shape assists with catching on a part of the implant when the capture device is retracted in a proximal direction.

FIG. 4B shows an embodiment of a distal end 410 which has an expansion slot 412 disposed therein, to allow for radial expansion, such as shown in FIG. 4C. In some embodiments, the distal end 410 returns to its original shape and/or dimensions after expanding. In some embodiments, the distal end 410 does not return to its original shape and/or dimensions after expanding. In some embodiments, the expansion slot 412 is not actually an opening or gap but an expanding section of the distal end 410, for example a zig-zag, accordion-like configuration which expands when urged outwardly. In some embodiments, this configuration is used because it allows the use of existing and/or legacy delivery systems.

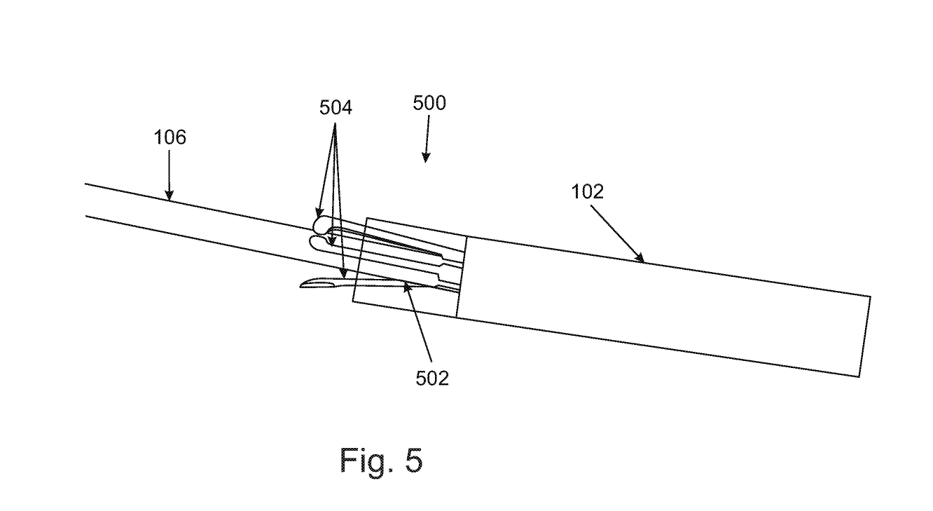

FIG. 5 is a side view of an implant retrieval system 500 with a capture device 502 partially within a catheter 102, in accordance with an exemplary embodiment of the invention. From this view, it can be seen that the capture arms 504 of the capture device 502 are still partially compressed/closed for navigation in the patient's body, but the arms 504 are also partially expanding as they are leaving the catheter 102. An inner catheter 106 is also shown in FIG. 5.

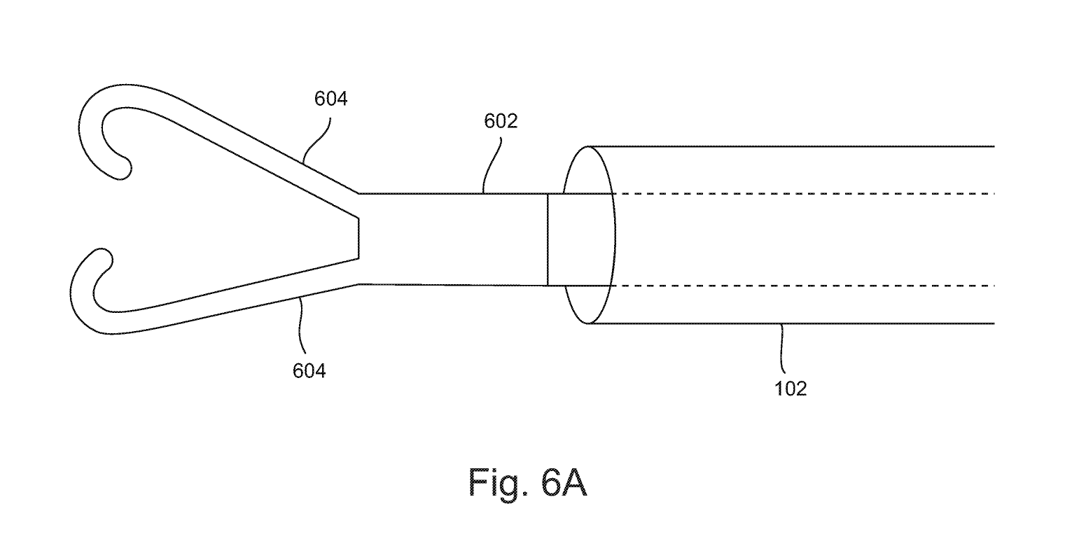

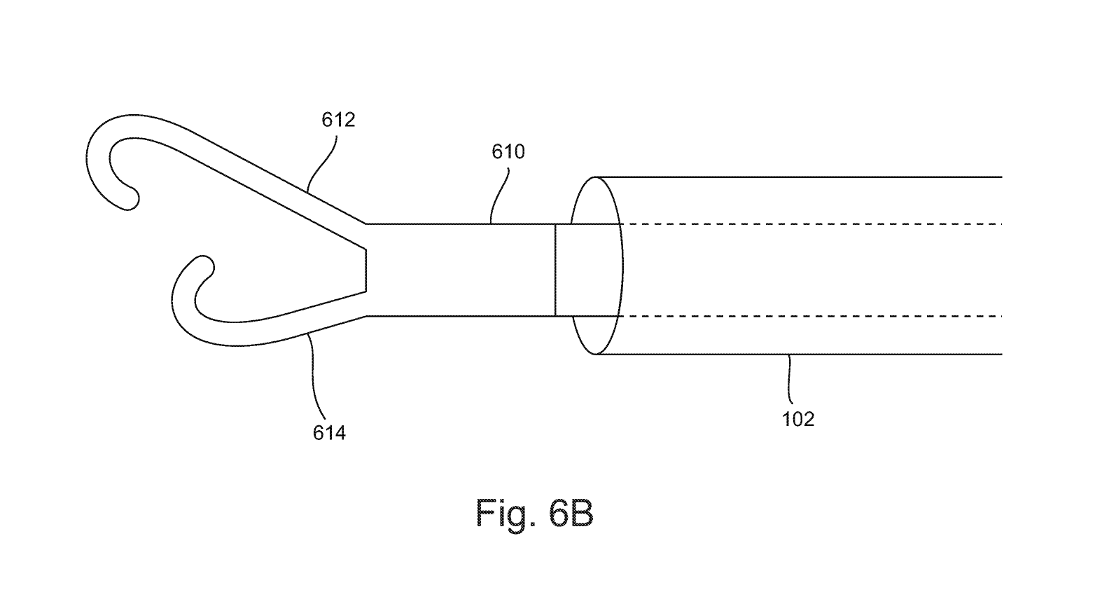

FIGS. 6A-6B are side views of different, deployed capturing arm configurations of an implant retrieval system 600, in accordance with exemplary embodiments of the invention. FIG. 6A shows a capture device 602 deployed from an outer catheter 102 where the arms 604 have expanded to retrieve an implant. In this embodiment, the arms 604 are substantially similar in configuration and the hooks face each other (i.e. they face in opposite directions, towards each other). FIG. 6B shows a capture device 610 deployed from an outer catheter 102 where a first arm 612 is longer than a second arm 614.

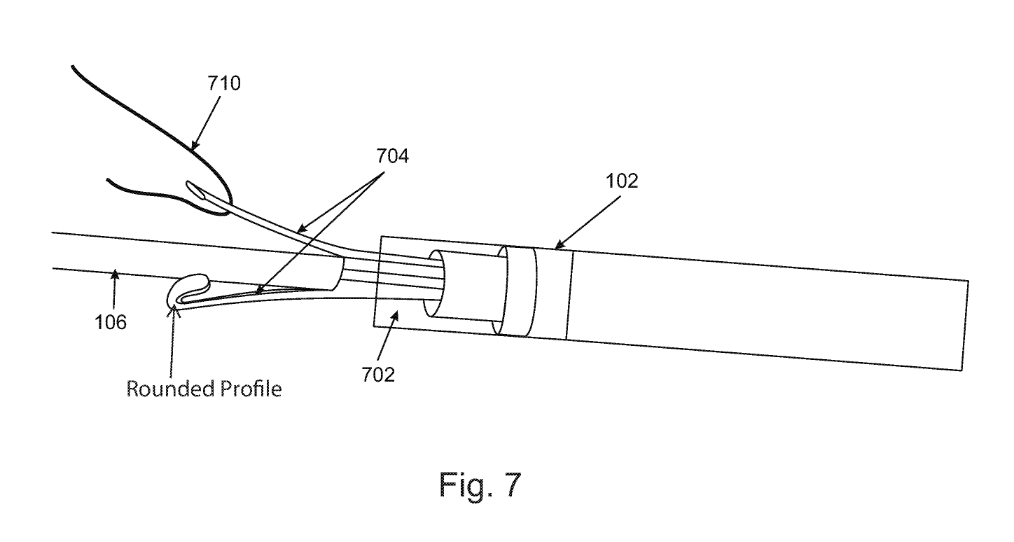

FIG. 7 is a close-up view of a portion of an implant 710 hooked by a capturing arm 704 of a capture device 702, in accordance with an exemplary embodiment of the invention.

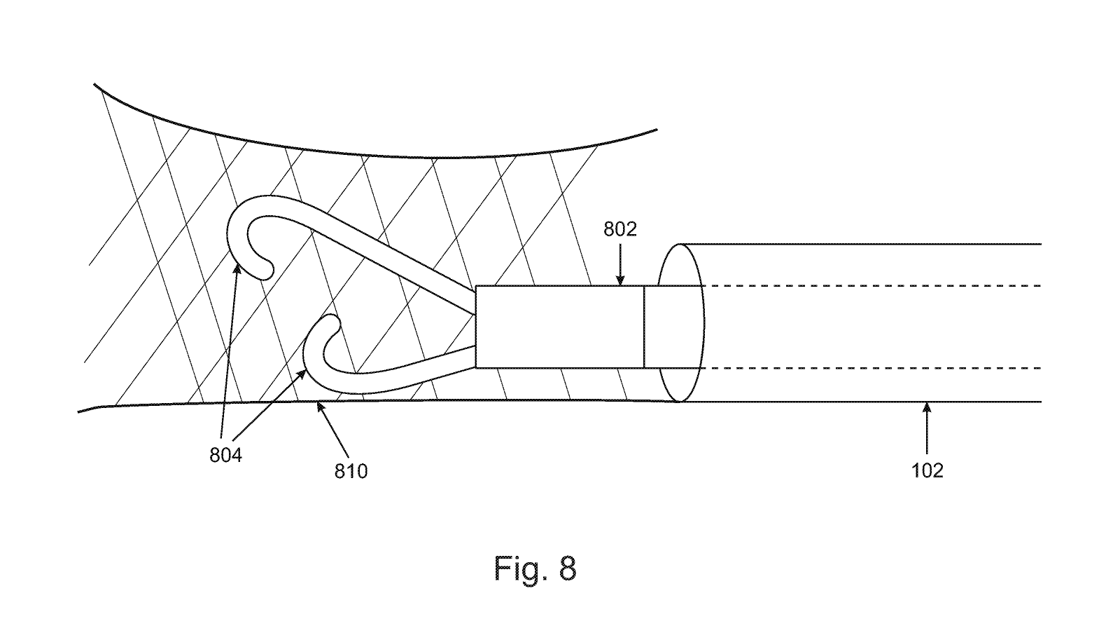

FIG. 8 is a close-up view of a portion of an implant 810 hooked by at least one capturing arm 804 of a capture device 802, in accordance with an exemplary embodiment of the invention. In an embodiment of the invention, at least one capturing arm 804 reversibly attaches to the implant 810 being retrieved, and in FIG. 8, two capturing arms 804 are shown hooking the implant 810. It should be understood that more than two arms could be used with capture device 802, and one some or all of the arms can have varied configurations. Further, depending on the scenario, such as arm configurations and/or the implant configuration and/or the implant location, one, some or all of the capturing arms will be employed to capture the implant, while one or some of the capturing arms may not. In some embodiments, the implant being retrieved is constructed of wire and/or has “petals” or loops which can be hooked or latched. In some exemplary embodiments of the invention, these loops or other aperture sections extend into a volume defined by the implant or away form a surface of an implant. For example, if implanted in a lumen, such loops or hooks or other aperture sections may extend between 1 and 20 mm into a body of said lumen, while part of the implant remains in contact with the lumen walls. In some exemplary embodiments of the invention, implant 810 is a stent-like implant which lies substantially flat against the lumen walls and arms 804 may be inserted between the implant and the wall, to effect hooking.

In some exemplary embodiments of the invention, arms 804 are so shaped that they can be “fiddled” against the implant, for example, rotated, advanced and retracted, without damaging luminal wall. For example, all outwardly pointing elements and/or all forward pointing elements, may be rounded, for example, having a radius of curvature of at least 0.2 mm, 0.5 mm, 1 mm, 2 mm or more. Optionally, the use of a planar material rather than wire for arms 804 provides stiffness suitable for pulling, while allowing more flexibility in directions not associated with retraction forces. Optionally, the distance of the point of contact of the implant with the capturing arm, once captured, and the axis of retraction force application is less than 3 mm, 2 mm 1 mm, 0.5 mm or smaller or intermediate distances. This may prevent flexing or twisting or other distortion of the capture arms during retraction, which distortion might otherwise release the captured implant. Optionally, the distance is configured to match desired pulling forces (e.g., above a certain force distortion may be desirable to avoid applying too much force on the implant). Optionally, the stiffness and geometry of arms 804 are selected to provide reliable retraction over a desired range of forces. In some exemplary embodiments of the invention, the arm stably captures the implant for retraction forces of up to 5 gr, 10 gr, 100 gr, 300 gr, 500 gr, 1 Kg, 2 Kg or smaller or intermediate forces, depending, for example, on the body organ from which the implant is being retrieved (e.g., soft, thin tissue or bone). Optionally, above such forces, arm 804 collapses, optionally elastically, and the captured implant is released.

In some exemplary embodiments of the invention, the depth of the recess in arms 802 which captures an implant is 0.5 mm, 1 mm, 2 mm, 3 mm, 4 mm, 5 mm or smaller or intermediate or greater depths. Such depths may be useful to tradeoff the amount of advancement needed to hook implant 802 with a resistance to elastic “popping out” of the hooked implant. Optionally, the recess is surrounded to a circumferential extent of at least 90 degrees, 120, degrees, 180 degrees, 200 degrees, 270 degrees, 300 degrees 350 degrees or smaller or intermediate degrees of surrounding (e.g., by arm 802 and/or extensions thereof). Optionally, arms 802 defines a helical shape of more than 360 degrees, with the capture recess inside. The degree of surrounding may be used to determine how easily implant 802 can slip off of the hooking arm, for example, to avoid tissue damage if too high a retraction force is applied.

In some exemplary embodiments of the invention, capturing of the implant provides a tactile feedback to the operator and/or can be seen on an imaging system, as the implant reacts to movement of device 802.

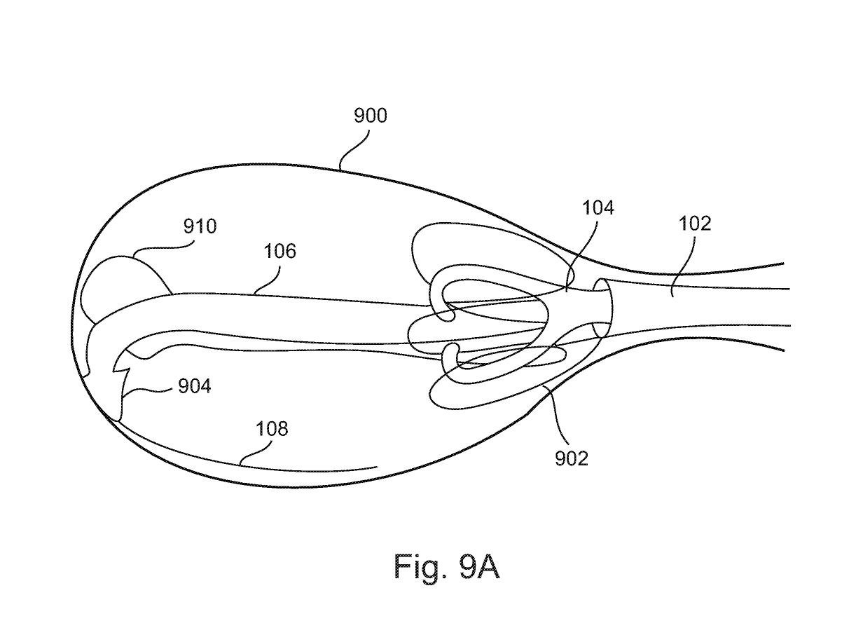

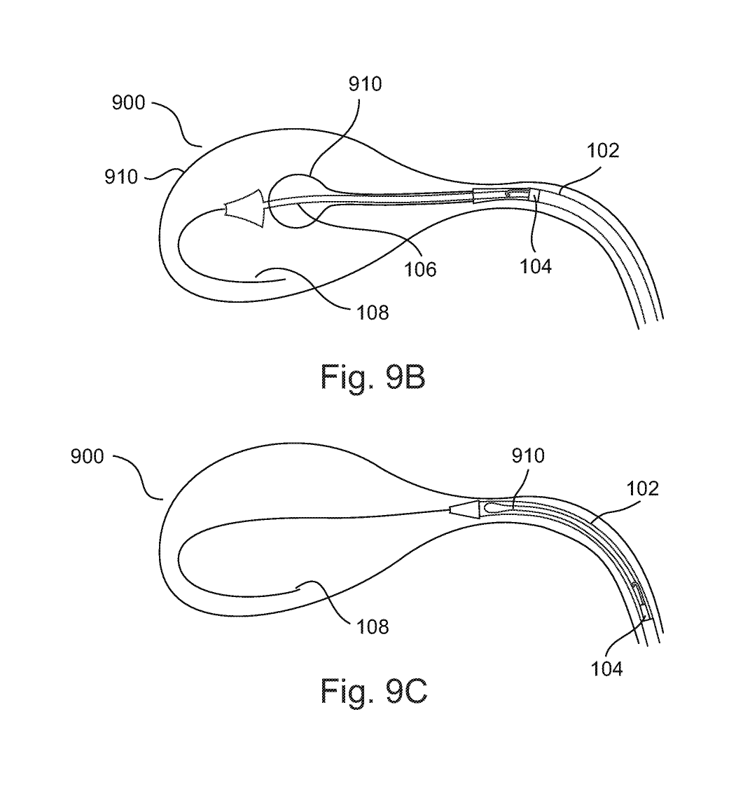

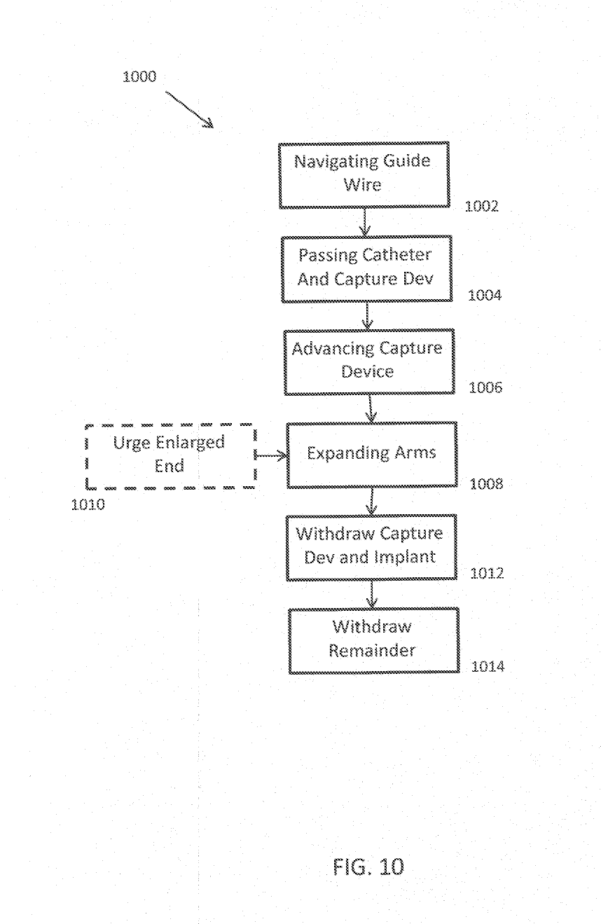

FIGS. 9A-9C show, graphically and sequentially, a method of retrieving an implant 910 from a gallbladder 900 using a capture device 102, in accordance with an exemplary embodiment of the invention. For efficiency, the flowchart 1000 of a method of retrieving an implant 910 from a gallbladder 900 is described in conjunction with FIGS. 9A-9C. In an embodiment of the invention, a guide wire 108 is navigated (1002) to the gallbladder 900 using conventional medical treatment-related navigational methods, possibly including medical imaging. Once the guide wire 108 is navigated (1002) to the proper implant retrieval location in the patient's body, at least outer catheter 102 and capture device 104 are passed (1004) into the patient over the guide wire 108 to the implant retrieval location, in this case in the gall bladder 900. In an embodiment of the invention, the capture device 104 is advanced (1006) out of the end of the outer catheter 102 upon arrival at the implant retrieval location in order to capture the implant to be retrieved. In some embodiments, the capturing arms 902 of the capture device 104 expand (1008) automatically when the device 104 is advanced out of the outer catheter 102 and/or the outer catheter 102 is pulled proximally to expose (1006) the capturing arms 902. In some embodiments, an inner catheter 106 with an enlarged end 904 is used to expand (1008) the capturing arms 404, by urging (1010) the enlarged end 904, originally disposed proximally in relation to the capturing arms 404, in the distal direction past the capturing arms 404, such that the enlarged end 904 passes the capturing arms 404 mechanically forcing them to expand (1008) outwardly or radially. In some embodiments, the ergonomically-shaped (is cone shaped and/or gradually increases in diameter and/or size) enlarged end 904 remains outside the outer catheter 102 and is used to assist with the navigating (1002) of the patient's body lumen to the implant retrieval location.

In an embodiment of the invention, the capture device 104 is advanced (1006) further to engage the implant 910 with at least one capturing arm 404. FIG. 9B shows the capture device 104 being withdrawn (1012) into the outer catheter 102 to retract the capture arms 404 and collapse the implant 910 into the lumen of the outer catheter 102.

FIG. 9C shows the capture device 104 withdrawn (1012) even further into the outer catheter, such that the implant 910 is fully enclosed by the outer catheter 102. In an embodiment of the invention, the capture device will be withdrawn (1012) all the way out of the outer catheter 102 on the proximal end (closest to an attending medical professional performing the procedure) with the implant 910 still attached to the capture device 104. The remaining structures, such as the guide wire and the inner catheter 106 (if used) are also withdrawn (1014), together or separately from the capture device 104 withdrawal (1012).

In some embodiments, the implant is retrieved in order to remove the debris (e.g. gall stones) collected by the implant. Optionally, vacuum is applied during the retrieval procedure to reduce undesirable debris flow into the body and/or to collect debris collected by the implant which comes loose during the retrieval process.

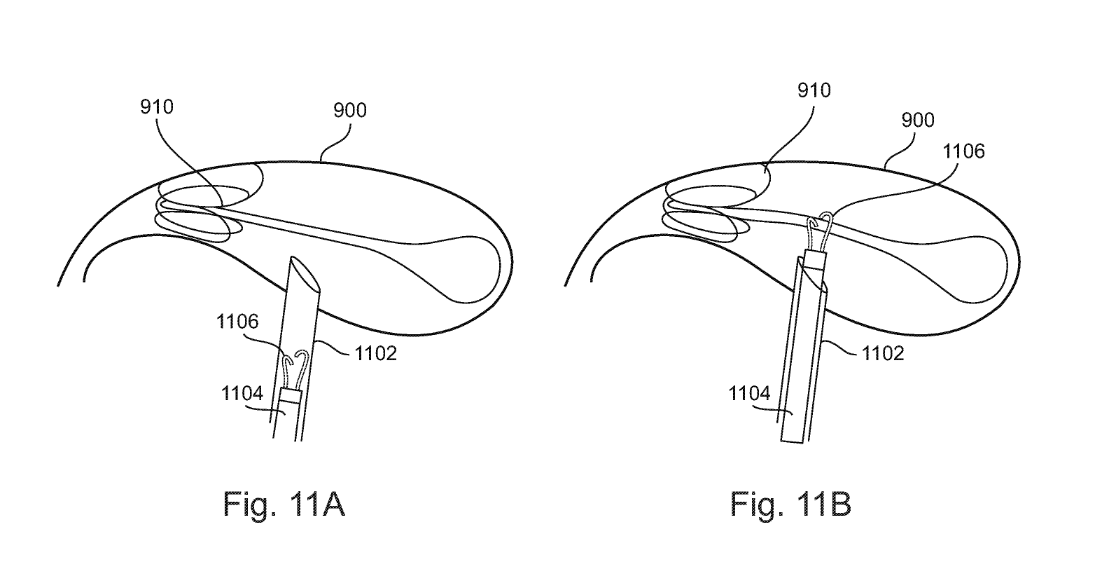

FIGS. 11A and 11B depict a method of retrieving a gallbladder implant 910 through a needle 1102, in accordance with an exemplary embodiment of the invention. FIG. 11A shows the needle 1102 being inserted through the wall of the gallbladder 900 with an implant capturing device 1104 located within the needle 1102. In some embodiments, the needle 1102 is preloaded with the capturing device 1104 before insertion into the patient. In some embodiments, the capturing device 1104 is inserted into the needle 1102 after the needle 1102 has penetrated the gallbladder 900 (or whatever other body lumen from which the implant 910 will be retrieved).

FIG. 11B shows the capturing device 1104 being deployed from the needle 1102, where the capturing arms 1106 of the capturing device 1104 engage with the implant 910 being retrieved. In an embodiment, the implant 910 is pulled in a proximal direction once the capturing arms 1106 have engaged with the implant 910, and the implant 910 is compressed into the lumen of the needle and withdrawn out of the patient's body.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Further, described ranges are intended to include numbers outside any range described within statistical error and/or inherent measurement equipment limitations.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. An implant retrieval system for retrieving a previously deployed implant in a patient's body lumen, comprising:
an outer catheter with a lumen disposed therethrough along a central axis of the outer catheter and configured with a diameter smaller than a diameter of the deployed implant; and,
a capture device, with a lumen disposed therethrough, configured with a smaller diameter than the outer catheter and configured to slide within the lumen of the outer catheter and including at least one capturing arm, outwardly and automatically expandable from the central axis and configured with an unexpanded diameter smaller than the diameter of the outer catheter, located at a distal end of the capture device,
wherein the at least one capturing arm engages the previously deployed implant for withdrawing the implant into the outer catheter, while the diameter of the outer catheter, which is smaller than the deployed implant, causes the implant to collapse while being withdrawn by the capture device into the outer catheter.

2. An implant retrieval system according to claim 1, further comprising an inner catheter configured to slide within the lumen of the capture device.

3. An implant retrieval system according to claim 2, further comprising a guide wire configured to slide within a lumen of the inner catheter.

4. An implant retrieval system according to claim 1, wherein the at least one capturing arm has a rounded hook shape configured to latch onto the implant.

5. An implant retrieval system according to claim 4, comprising a plurality of capturing arms each face in the same direction.

6. An implant retrieval system according to claim 4, comprising a plurality of capturing arms, at least one capturing arm facing in an opposite direction from at least one other capturing arm of the plurality of capturing arms.

7. An implant retrieval system according to claim 1, wherein the at least one capturing arm is configured with at least one of a rounded, curved and flat shape.

8. An implant retrieval system according to claim 1, wherein at least one capturing arm is longer than another at least one other capturing arm.

9. An implant retrieval system according to claim 1, wherein the distal end of the capture device is configured with an expansion slot configured to enable the radial expansion of the distal end.

10. An implant retrieval system according to claim 2, wherein an enlarged end is disposed on a distal end of the inner catheter.

11. An implant retrieval system according to claim 10, wherein the enlarged end is conical shaped.

12. An implant retrieval system according to claim 10, wherein the enlarged end is disposed on the inner catheter proximally of the at least one capturing arm such that advancement of the inner catheter distally causes the enlarged end to mechanically and reversibly expand the at least one capturing arm outward from the central axis.

13. An implant retrieval system according to claim 10, wherein the enlarged end has a diameter at least as large as the outer catheter.

14. An implant retrieval system according to claim 1, wherein the capturing device is constructed of an elastic material.

15. An implant retrieval system according to claim 1, wherein the implant retrieval system is at least 5 F.

16. An implant retrieval system according to claim 1, wherein the implant retrieval system is between 7 F and 8.5 F.

17. A method for retrieving a previously deployed implant from a patient, comprising:
navigating an implant retrieval system including an outer catheter and a capture device to the previously deployed implant, wherein the capture device is configured with a lumen disposed therethrough, configured with a smaller diameter than the outer catheter and configured to slide within the lumen of the outer catheter;
exposing at least one capturing arm of the capture device, wherein the at least one capturing arm is outwardly and automatically expandable from the central axis and configured with an unexpanded diameter smaller than the diameter of the outer catheter, located at a distal end of the capture device;
latching the at least one exposed capturing arm onto the previously deployed implant while also collapsing the implant as the implant is drawn into the outer catheter; and,
withdrawing the previously deployed implant and the capture device from the patient.

18. A method according to claim 17, wherein the at least one capturing arm automatically expands when exposed by pulling back the outer catheter.

19. A method according to claim 17, further comprising mechanically enlarging the at least one capturing arm of the capture device outwardly or radially using an enlarged end of an inner catheter disposed in a lumen of the capture device.

20. A method according to claim 17, further comprising withdrawing from the patient the remainder of the implant retrieval system and the previously deployed implant and the capture device.
